(19) United States
(12) Patent Application Publication
(10) Pub. No.: US 2024/0016886 A1
(43) Pub. Date: Jan. 18, 2024

(54) USE OF ANNEXIN V TO IMPROVE THE PURITY, SOLUBILITY AND LONG-TERM STABILITY OF PHAGE SUSPENSIONS

(71) Applicant: The Board of Trustees of the Leland Stanford Junior University, Stanford, CA (US)

(72) Inventors: Francis Gerard Blankenberg, Portola Valley, CA (US); Derek Holman, Redwood City, CA (US)

(21) Appl. No.: 18/036,602

(22) PCT Filed: Nov. 16, 2021
(86) PCT No.: PCT/US2021/059580

§ 371 Date: May 11, 2023

Related U.S. Application Data
(60) Provisional application No. 63/115,424, filed on Nov. 18, 2020.

(51) Int. Cl. A61K 38/17; A61K 35/76; A61P 31/04

(52) U.S. Cl. CPC A61K 38/17; A61K 35/76; A61P 31/04

(57) ABSTRACT

Methods are provided for the prevention of aggregation, simple purification stabilization and preservation of phage preparations for the use of treating bacterial infections.

### CROSS-REFERENCE TO RELATED APPLICATIONS

This application claims the benefit of U.S. provisional Patent Application Ser. No. 63/115,424, filed on Nov. 18, 2020, the contents of which are herein incorporated by reference in their entirety.

### INCORPORATION BY REFERENCE OF SEQUENCE LISTING PROVIDED AS A TEXT FILE

A Sequence Listing is provided herewith in a text file, (STAN-1791WO_SeqListing_ST25), created on Nov. 16, 2021, and having a size of 4000 bytes. The contents of the text file are incorporated herein by reference in its entirety.

### BACKGROUND

The increasing threat of antibiotic resistance has reignited interest in the use of phages, which are natural predators of bacteria, for the treatment of multidrug-resistant bacterial infections. However, despite human exposure to phages from birth and continuously throughout life, the safety and efficacy of phage preparations in humans beyond the skin and external ear are currently unknown. A confounding variable in the testing of phage has been the frequent use of bacterial lysates or incompletely purified phage preparations. Additionally, the mechanisms of bacterial resistance to phage can vary widely, for example including the modification of cell-surface receptors, restriction-modification of incoming phage DNA, and interphage interference. Another factor is the high rate of spontaneous bacterial-resistance mutations to phage observed in vitro, which are likely to be selected in vivo. As phages can also be efficient transmitters of genetic material, there are potential clinical risks associated with frequent exposure of host bacteria to phage in vivo, or the use of phage cocktails to broaden bacterial coverage.

The field lacks generalizable methods to consistently generate highly concentrated, contaminant-free bacterial phage suspensions that are stable in solution without self-aggregation or loss of viability, for systemic injection. The present disclosure provides improved methods for the prevention of aggregation, simple purification stabilization and preservation of phage preparations for the use of treating bacterial infections.

### SUMMARY

Formulations and methods are provided for the preparation and stabilization of high concentration phage stocks and/or cultures, where such stocks and/or/cultures are stabilized to have reduced bacterial contamination, phage aggregation, and phage precipitation, relative to non-stabilized formulations. In some embodiments, the high concentration phage preparations are used in a treatment for a bacterial infection. The formulations comprise annexin to stabilize the high concentration phage preparations. The method and formulations allow for the treatment of bacterial infections through lysis of bacterial membranes, which requires using high concentration of phage.

Phage preparations with greater than about 10¹² pfu/ml can achieve local phage concentrations that are capable of overwhelming a bacterial population in a process called, “lysis from without”. Lysis from without, or direct phage lysis, occurs by the simultaneous direct interaction of many phages with the outer bacterial membrane. Although lysis from without is expected to be rare and uncommon in vivo with standard phage injection densities of from about 10⁷-10⁸ pfu/ml, a single dose of phage at concentrations greater than about 10¹² pfu/ml can rapidly reverse lethal bacterial wound infections in mice. The advantages of direct phage lysis include, speed, reduced chance of resistant gene transmission avoidance of known bacterial phage resistance mechanisms, elimination of interphage interference, and the potential increase in phage cross-reactivity, which could broaden bacterial coverage without the use of phage cocktails.

In some embodiments, a high concentration phage formulation comprises greater than about 10⁸ pfu/ml. In some embodiments, high concentration phage formulation comprises from about 10⁸-10⁹ pfu/ml, 10⁹-10¹⁰ pfu/ml, 10¹⁰-10¹¹ pfu/ml, 10¹⁰-10¹² pfu/ml, or 10¹²-10¹³. In some embodiments, a high concentration phage formulation comprises greater than about 10¹⁰ pfu/ml, greater than about 10¹¹ pfu/ml, greater than about 10¹² pfu/ml or greater than about 10¹³ pfu/ml.

In some embodiments, the phage is a filamentous phage. Examples of filamentous phage species (spp.) include, but are not limited to, Vibrio virus CTXphi, Propionibacterium virus B5, Vibrio virus KSF1, Xanthomonas virus Cf1c, Vibrio virus fs1, Vibrio virus VGJ, Ralstonia virus RS551, Ralstonia virus RS603, Ralstonia virus RSM1, Ralstonia virus RSM3, Escherichia virus If1, Escherichia virus M13, Escherichia virus 122, Salmonella virus Ike, Ralstonia virus PE226, Pseudomonas virus Pf1, Pseudomonas virus Pf3, Pseudomonas virus Pf4, Pseudomonas virus PAML31-1, Stenotrophomonas virus PSH1, Ralstonia virus RSS1, Vibrio virus fs2, Vibrio virus VFJ, Stenotrophomonas virus SMA6, Stenotrophomonas virus SMA9, Stenotrophomonas virus SMA7, Thermus virus OH3, Vibrio virus VfO3K6, Vibrio virus VCY, Vibrio virus Vf33, Xanthomonas virus Xf109, etc.

In some embodiments, the stabilizing annexin is human annexin V. In some embodiments, the amount of annexin used to stabilize a high concentration phage formulation is about 0.01 mg/ml, 0.02 mg/ml, 0.04 mg/ml, 0.06 mg/ml, 0.08 mg/ml, 0.1 mg/ml, 0.2 mg/ml, 0.4 mg/ml, 0.6 mg/ml, 0.8 mg/ml, 1 mg/ml or greater than 1 mg/ml. In some embodiments, Ca⁺⁺ is used, for example from about 1 mM to about 5 mM ionized Ca⁺⁺, in addition to annexin, to allow for high concentration phage preparation. In some embodiments, DNAse I is used in conjunction with annexin and/or Ca⁺⁺ in phage preparations. Stabilization with annexin can reduce the level of bacterial contamination to at least <0.01% of the total solution. In some embodiments, the level of bacterial contamination is from about 0.01-0.005%, 0.005-0.001%, 0.001-0.0005%, 0.0005-0.0001%, or <0.0001% wt/wt. Stabilization with annexin can also reduce phage precipitation by at least 10% relative to unstabilized preparations. In some embodiments, phage precipitation is reduced by 10-20%, 20-30%, 30-40%, 40-50% or >50% using annexin.

In some embodiments, a bacterial infection that is treated with a high concentration phage formulation is caused by gram-positive bacteria. Examples of gram-positive bacteria include, but are not limited to, Staphylococcus spp., Streptococcus spp., Listeria spp., Bacillus spp., Clostridium spp., Corynebacterium spp., Enterococcus spp., Mycobacterium spp., Propionibacterium spp., etc.

In some embodiments, a bacterial infection that is treated with a high concentration phage formulation is caused by gram-negative bacteria. Examples of gram-negative bacteria include, but are not limited to, Pseudomonas spp., Escherichia spp., Helicobacter spp., Salmonella spp., Legionella spp., Vibrio spp., Shigella spp., Enterobacter spp., Neisseria spp., Klebsiella spp., Yersinia spp., Acinetobacter spp., Proteus spp., Bartonella spp., Bordetella spp., Campylobacter spp., Haemophilus spp., Treponema spp., Chlamydia spp., etc.

In some embodiments, a bacterial infection that is treated with a high concentration phage formulation is caused by antibiotic resistant bacteria. Examples of antibiotic resistant bacteria include, but are not limited to, Pseudomonas aeruginosa, Escherichia coli, Staphylococcus aureus, Acinetobacter baumannii, Clostridium difficile, Klebsiella pneumoniae, Neisseria gonorrhoeae, Campylobacter jejuni, Enterococcus faecium, Salmonella Typhi, nontyphoidal Salmonella, Shigella dysenteriae, Shigella flexneri, Shigella boydii, Shigella sonnei, Streptococcus pneumoniae, Mycobacterium tuberculosis, etc.

In some embodiments, a bacterial infection that is treated with a high concentration phage formulation is a local infection at the site of a wound or implanted device. In other embodiments, the bacterial infection is a systemic infection. In some embodiments, a high concentration phage formulation is delivered directly to the site of infection. In other embodiments, a high concentration phage formulation is administered systemically. In some embodiments, the formulation is administered in a bolus injection. In other embodiments the formulation is administered in a manner that provides for prolonged blood clearance of the phage, for example where the half-life of the phage in circulation is at least about 30 minutes, at least about 1 hour, at least about 1.5 hours, at least about 2 hours, at least about 2.5 hours, at least about 3 hours or more. In other embodiments the route of administration is intra-venous injection over an extended period of time, for example where a daily dosage is delivered over a period of up to 30 minutes, up to one hour, up to 2 hours, up to 4 hours, up to 6 hours, up to 8 hours, up to 12 hours, up to 16 hours, up to 24 hours.

### BRIEF DESCRIPTION OF DRAWINGS

FIG. 1 Distribution of radiolabeled phage in mouse organs following bolus injection.

FIG. 2 Total urinary excretion of radiolabeled phage following bolus injection.

FIG. 3 Distribution of radiolabeled phage following bolus injection. Rapid clearance of ⁹⁹mTc-HYNIC phage from the blood and lungs, while the uptake of radiolabeled phage in other major organs, including the liver, spleen, kidneys, and bone marrow (femur) remained relatively constant after 30 min.

FIG. 4 ⁹⁹mTc-phage SPECT Imaging of Pseudomonas Wound Infection. Four mice were wounded and infected with 1×10⁶ cfu of Xen5 bacteria on Day 0. On Day 1, mice were injected IV with 20 MBq (550 μCi) of radiolabeled phage; two hours later, they underwent SPECT imaging. Multifocal uptake of phage occurred within the actual wound and the deeper tissues of the back and neck in mice 1, 3, and 4. Mouse 2 had little visible uptake of radiolabeled phage. Wound-related uptakes for mice 1, 2, 3, and 4 were 3.5, 1.4, 15.8, and 5.9, (target/background), respectively.

FIG. 5 Bioluminescent imaging of a mouse infected with Pseudomonas aeruginosa. Despite a single focus of BLI signal within the wound, SPECT imaging showed extensive uptake within the deep back fascia and fat, as well as in the neck and peri-wound region, suggesting bacterial spread to local soft tissues and the lymphatic system well beyond that detectable by BLI.

FIG. 6 Serial bioluminescent imaging of mice infected with Pseudomonas aeruginosa. The BLI signal was promptly reduced in all four mice the day after radiolabeled phage injection, which progressively decreased on days 3 and 4.

FIG. 7 Annexin V Reduces Phage Aggregation as seen with DLS. Samples of LMA2/Myoviridae, PB-1-like (A & B), LUZ24/Podoviridae, T7-like (C & D) and LUZ14/Podoviridae, T7-like (E & F) short tailed phages capable of infecting and lysing clinically isolated common strains of Pseudomonas aeruginosa.

### DETAILED DESCRIPTION

### Definitions

Before embodiments of the present disclosure are further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Any methods and materials similar or equivalent to those described herein can also be used in the practice or testing of embodiments of the present disclosure.

It must be noted that as used herein and in the appended claims, the singular forms “a”, “and”, and “the” include plural referents unless the context clearly dictates otherwise. Thus, for example, reference to “a compound” includes not only a single compound but also a combination of two or more compounds, reference to “a substituent” includes a single substituent as well as two or more substituents, and the like.

In describing and claiming the present invention, certain terminology will be used in accordance with the definitions set out below. It will be appreciated that the definitions provided herein are not intended to be mutually exclusive. Accordingly, some chemical moieties may fall within the definition of more than one term.

As used herein, the phrases “for example,” “for instance,” “such as,” or “including” are meant to introduce examples that further clarify more general subject matter. These examples are provided only as an aid for understanding the disclosure, and are not meant to be limiting in any fashion.

Gram-negative bacteria. Gram-negative bacteria are a specific type of bacteria with unique characteristics. Like most bacteria, they can cause infections throughout the body. Common infection sites include the lungs, urinary tract, bloodstream, nervous system, and soft tissues. Surgical wounds can also become infected with gram-negative bacteria. Common gram-negative bacteria and the infections they cause include Escherichia coli (E. coli), which cause food poisoning, urinary tract infections, gastroenteritis, and newborn meningitis; Pseudomonas aeruginosa, which cause lung and urinary tract infections; Klebsiella, which cause meningitis, and lung, urinary tract, and bloodstream infections; Acinetobacter baumannii, which cause several types of infections in wounded soldiers; Neisseria gonorrhoeae, which cause gonorrhea, a sexually transmitted disease; Enterobacteriaceae, which cause urinary tract, lung, and bloodstream infections, and food poisoning (includes carbapenem-resistant Enterobacteriaceae, which are very resistant to antibiotics).

Some of the more problematic drug-resistant pathogens encountered today include methicillin-resistant Staphylococcus aureus, multidrug-resistant Streptococcus pneumoniae, and vancomycin-resistant Enterococcus spp., multi-drug resistant Mycobacterium tuberculosis, Clostridium difficile, and Streptococcus pneumoniae among the gram-positive bacteria and multidrug-resistant Acinetobacter baumannii, Klebsiella pneumoniae, Escherichia coli, Campylobacter jejuni, Neisseria gonorrhoeae, Salmonella Typhi, nontyphoidal Salmonella, Shigella dysenteriae, Shigella flexneri, Shigella boydii, Shigella sonnei, and Pseudomonas aeruginosa among the gram-negative bacteria.

P. aeruginosa is a ubiquitous organism present in many diverse environmental settings, and it can be isolated from various living sources, including plants, animals, and humans. The ability of P. aeruginosa to survive on minimal nutritional requirements and to tolerate a variety of physical conditions has allowed this organism to persist in both community and hospital settings. In the hospital, P. aeruginosa can be isolated from a variety of sources, including respiratory therapy equipment, antiseptics, soap, sinks, mops, medicines, and physiotherapy and hydrotherapy pools. Community reservoirs of this organism include swimming pools, whirlpools, hot tubs, contact lens solution, home humidifiers, soil and rhizosphere, and vegetables.

P. aeruginosa presents a serious therapeutic challenge for treatment of both community-acquired and nosocomial infections. The use of an appropriate antibiotic is complicated by the ability of P. aeruginosa to develop resistance to multiple classes of antibacterial agents, even during the course of treating an infection. Epidemiological outcome studies have shown that infections caused by drug-resistant P. aeruginosa are associated with significant increases in morbidity, mortality, need for surgical intervention, length of hospital stay and chronic care, and overall cost of treating the infection. Even more problematic is the development of resistance during the course of therapy, a complication which has been shown to double the length of hospitalization and overall cost of patient care. P. aeruginosa can develop resistance to antibacterials either through the acquisition of resistance genes on mobile genetic elements (i.e., plasmids) or through mutational processes that alter the expression and/or function of chromosomally encoded mechanisms. Both strategies for developing drug resistance can severely limit the therapeutic options for treatment of serious infections.

Bacteriophages, also known as phages, are bacterial viruses that attach to their specific hosts and kill them by internal replication and bacterial lysis involving a complex lytic cycle involving several structural and regulatory genes. Phages are very specific in that they only attack their targeted bacterial hosts. The specificity of phages makes it possible to target only the pathogen responsible for the infection to be treated, and thus to preserve the commensal or mutualist bacteria that compose the microbiota, whose role in human health we are only just beginning to understand.

Phage therapy involves the targeted application of bacteriophages that, upon encounter with specific pathogenic bacteria, can infect and kill them. As typically practiced, phages then lyse those bacteria, releasing virion progeny that can continue the cycle, including migrating to other sites of infection anywhere in the body. The actual phage-mediated bacterial killing, however, occurs well prior to the lysis step, e.g., such as in the first minutes of infection for a phage such as phage T4, as the phage converts the cell into a factory for making new phages. Phages are unique among antibacterial agents in their ability to increase their numbers when in the presence of bacterial targets. Of similar importance, phages only minimally impact non-target bacteria or body tissues.

The terms “active agent,” “antagonist”, “inhibitor”, “drug” and “pharmacologically active agent” are used interchangeably herein to refer to a chemical material or compound which, when administered to an organism (human or animal) induces a desired pharmacologic and/or physiologic effect by local and/or systemic action.

As used herein, the terms “treatment,” “treating,” and the like, refer to obtaining a desired pharmacologic and/or physiologic effect, such as reduction of viral titer. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease (e.g., reduction in bacterial titers).

The terms “individual,” “host,” “subject,” and “patient” are used interchangeably herein, and refer to an animal, including, but not limited to, human and non-human primates, including simians and humans; rodents, including rats and mice; bovines; equines; ovines; felines; canines; avians, and the like. “Mammal” means a member or members of any mammalian species, and includes, by way of example, canines; felines; equines; bovines; ovines; rodentia, etc. and primates, e.g., non-human primates, and humans. Non-human animal models, e.g., mammals, e.g. non-human primates, murines, lagomorpha, etc. may be used for experimental investigations.

As used herein, the terms “determining,” “measuring,” “assessing,” and “assaying” are used interchangeably and include both quantitative and qualitative determinations.

The terms “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and native leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like.

The terms “nucleic acid molecule” and “polynucleotide” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

A “therapeutically effective amount” or “efficacious amount” means the amount of a compound that, when administered to a mammal or other subject for treating a disease, condition, or disorder, is sufficient to affect such treatment for the disease, condition, or disorder. The “therapeutically effective amount” will vary depending on the compound, the disease and its severity and the age, weight, etc., of the subject to be treated.

The term “unit dosage form,” as used herein, refers to physically discrete units suitable as unitary dosages for human and animal subjects, each unit containing a predetermined quantity of a compound calculated in an amount sufficient to produce the desired effect in association with a pharmaceutically acceptable diluent, carrier or vehicle. The specifications for unit dosage forms depend on the particular compound employed and the effect to be achieved, and the pharmacodynamics associated with each compound in the host.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical formulation that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes both one and more than one such excipient, diluent, carrier, and adjuvant.

As used herein, a “pharmaceutical formulation” is meant to encompass a formulation suitable for administration to a subject, such as a mammal, especially a human. In general a “pharmaceutical formulation” is sterile, and preferably free of contaminants that are capable of eliciting an undesirable response within the subject (e.g., the compound(s) in the pharmaceutical formulation is pharmaceutical grade). Pharmaceutical formulations can be designed for administration to subjects or patients in need thereof via a number of different routes of administration including oral, buccal, rectal, parenteral, intraperitoneal, intradermal, intracheal, intramuscular, subcutaneous, and the like.

Annexin-V (PAP-I, lipocortin-V) acts as a potent anticoagulant by binding to negatively charged phospholipids with high affinity, for example having a Kd in the 10⁻⁹ to 10⁻¹⁰ M range. Annexin V forms a shield around negatively-charged phospholipid molecules. The formation of blocks the entry of phospholipids into coagulation (clotting) reactions, and prevents interaction of the phospholipid with immunoregulatory cells. The genetic sequence of human annexin V can be accessed at Genbank, NM_001154. The crystal and molecular structure is described in Romisch and Paques (1992) J. Mol. Biol. 223 (3), 683-704. Annexin V polypeptides or biologically active fragments and variants thereof, and the like, are used in the treatment of cancer. In some embodiments the annexin V has a wild-type or native sequence.

The Annexin A5 or the functional analog or variant thereof according to the invention can be human Annexin A5 (SEQ ID NO:1), an allelic or genetic variant thereof, a mammalian orthologue thereof, or an allelic or genetic variant thereof. A functional analog or variant of Annexin A5 may be more than 50%, 60%, 70%, 75%, such as more than 80% or 85%, more than 90%, or more than 95% or 99% identical to human Annexin A5, SEQ ID NO:1. The Annexin V can lack a terminal methionine.

The functional analog or variant of Annexin A5 according to the invention may, or may not, be a dimer of Annexin A5 or a functional analog or variant thereof, or may or may not, be a PEGylated Annexin A5 or a functional analog or variant thereof. DiAnnexinA5 and PEGylated AnnexinA5 are disclosed in WO 02/067857.

PEGylation is a method well known to those skilled in the art wherein a polypeptide or peptidomimetic compound (for the purposes of the present invention, Annexin V or the functional analog or variant) is modified such that one or more polyethylene glycol (PEG) molecules are covalently attached to the side chain of one or more amino acids or derivatives thereof. It is one of the most important molecule altering structural chemistry techniques (MASC). Other MASC techniques may be used; such techniques may improve the pharmacodynamic properties of the molecule, for example extending its half life in vivo. A PEG-protein conjugate is formed by first activating the PEG moiety so that it will react with, and couple to, the protein or peptidomimetic compound of the invention. PEG moieties vary considerably in molecular weight and conformation, with the early moieties (monofunctional PEGs; mPEGs) being linear with molecular weights of 12 kDa or less, and later moieties being of increased molecular weights. PEG2, a recent innovation in PEG technology, involves the coupling of a 30 kDa (or less) mPEG to a lysine amino acid (although PEGylation can be extended to the addition of PEG to other amino acids) that is further reacted to form a branched structure that behaves like a linear mPEG of much greater molecular weight (Kozlowski et al., (2001), Biodrugs 15, 419-429). Methods that may be used to covalently attach the PEG molecules to polypeptides are further described in Roberts et al., (2002) Adv Drug Deliv Rev, 54, 459-476, Bhadra et al., (2002) Pharmazie 57, 5-29, Kozlowski et al., (2001) J Control Release 72, 217-224, and Veronese (2001) Biomaterials 22, 405-417 and references referred to therein.

The advantages of PEGylation to the polypeptide or peptidomimetic compound of the invention include reduced renal clearance which, for some products, results in a more sustained adsorption after administration as well as restricted distribution, possibly leading to a more constant and sustained plasma concentrations and hence an increase in clinical effectiveness (Harris et al., (2001) Clin Pharmacokinet 40, 539-551). Further advantages can include reduced immunogenicity of the therapeutic compound (Reddy, (2001) Ann Pharmacother 34, 915-923), and lower toxicity (Kozlowski et al., (2001), Biodrugs 15, 419-429).

The functional analog or variant of Annexin A5 according to the invention can be a fusion protein comprising the sequence of Annexin A5 or a variant thereof. Thus, for example, Annexin A5 or a variant thereof can be fused to one or more fusion partner polypeptide sequence(s) so as to extend the half-life of the molecule within a patient's circulatory system and/or add further functionality to the molecule.

By a “functional” analog or variant of Annexin A5 is meant a protein capable of binding to phosphatidylserine on a biological membrane, preferably to a level that is at least 10%, 20%, 30%, 40%, 50%, 60%, 70%, 80%, 90%, 95%, 99% or about 100% of that displayed by human Annexin A5 (SEQ ID NO:1) under the same conditions. Suitable method for measuring Annexin A5 binding to phosphatidylserine on a biological membrane are known in the art.

A “functional” analog or variant of Annexin A5 may, additionally, or alternatively, also possess at least 10%, 20%, 30%, 40%, 50%, 60%, 70%, 80%, 90%, 95%, 99% or about 100% of the therapeutic activity human Annexin V (SEQ ID NO:1) when used at the same (i.e. molar equivalent) dosage, for blocking or reversing EV mediated transcriptional changes.

Annexin V polypeptides, which can be used in the methods of the invention, comprise at least about 50 contiguous amino acids, usually at least about 100 contiguous amino acids, at least about 150 contiguous amino acids, at least about 200 contiguous amino acids, at least about 250 contiguous amino acids, and which may include up to the full length of native annexin V protein, including without limitation human annexin V protein, or modifications thereof, and may further include fusion polypeptides as known in the art in addition to the provided sequences.

A pharmaceutical composition comprises Annexin V or a functional analog or variant thereof in admixture with a pharmaceutically or veterinarily acceptable adjuvant, diluent or carrier, which will typically be selected with regard to the intended route of administration and standard pharmaceutical practice. The composition may be in the form of immediate-, delayed- or controlled-release applications. Preferably, the formulation is a unit dosage containing a daily dose or unit, daily sub-dose or an appropriate fraction thereof, of the active ingredient.

The pharmaceutical composition according to the invention may, or may not, be intended for, and, thus formulated in a manner suitable for, parenteral, intravenous, intra-arterial, intraperitoneal, intra-muscular or subcutaneous administration, or they may be administered by infusion techniques. They may be best used in the form of a sterile aqueous solution which may contain other substances, for example, enough salts or glucose to make the solution isotonic with blood. The aqueous solutions may be suitably buffered (preferably to a pH of from 3 to 9), if necessary. The preparation of suitable pharmaceutical formulations under sterile conditions is readily accomplished by standard pharmaceutical techniques well-known to those skilled in the art.

Such formulations may include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

A therapeutically effective amount of Annexin V or a functional analog or variant thereof for administration to a patient, such as a human patient, on the basis of a daily dosage level may be from 0.01 to 1000 mg of Annexin V or a functional analog or variant thereof per adult (for example, from about 0.001 to 20 mg per kg of the patient's body weight, such as 0.01 to 10 mg/kg, for example greater than 0.1 mg/kg and less than 20, 10, 5, 4, 3 or 2 mg/kg, such as about 1 mg/kg), administered in single or divided doses.

The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention.

The annexin V for use in the subject methods may be produced from eukaryotic or prokaryotic cells, or may be synthesized in vitro. Where the protein is produced by prokaryotic cells, it may be further processed by unfolding, e.g. heat denaturation, DTT reduction, etc. and may be further refolded, using methods known in the art. By using synthesizers, naturally occurring amino acids may be substituted with unnatural amino acids. The particular sequence and the manner of preparation will be determined by convenience, economics, purity required, and the like.

Modifications of interest that do not alter primary sequence include chemical derivatization of polypeptides, e.g., acylation, acetylation, carboxylation, amidation, etc. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine.

If desired, various groups may be introduced into the peptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

The polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

In one embodiment of the invention, the Annexin V polypeptide consists essentially of a polypeptide sequence of around about 320 amino acids in length and having a sequence of a native Annexin V protein, or an Annexin V protein lacking a terminal methionine as described above. By “consisting essentially of” in the context of a polypeptide described herein, it is meant that the polypeptide is composed of the Annexin V sequence, which sequence is optionally flanked by one or more amino acid or other residues that do not materially affect the basic characteristic(s) of the polypeptide.

A reference sequence of human Annexin V has a sequence as follows:

(SEQ ID NO: 1)
MAQVLRGTVTDFPGFDERADAETLRKAMKGLGTDEESILTL
LTSRSNAQRQEISAAFKTLFGRDLLDDLKSELTGKFEKLI
VALMKPSRLYDAYELKHALKGAGTNEKVLTEIIASRTPEE
LRAIKQVYEEEYGSSLEDDVVGDTSGYYQRMLVVLLQANR
DPDAGIDEAQVEQDAQALFQAGELKWGTDEEKFITIFGTR
SVSHLRKVFDKYMTISGFQIEETIDRETSGNLEQLLLAVV
KSIRSIPAYLAETLYYAMKGAGTDDHTLIRVMVSRSEIDL
FNIRKEFRKNFATSLYSMIKGDTSGDYKKALLLLCGEDD.

As used herein, the term “administration” refers to the administration of a formulation (i.e. a stabilized phage formulation) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (including by intratracheal instillation), transdermal, vaginal and vitreal. In some embodiments, administration may involve intermittent dosing. In some embodiments, administration may involve continuous dosing (e.g., perfusion) for at least a selected period of time. As is known in the art, antibody therapy is commonly administered parenterally (e.g., by intravenous or subcutaneous injection).

Pharmaceutical Formulations

Annexin stabilized high concentration page formulations can be generated by purifying phage in the presence of annexin. Optionally, the phage are initially grown by infection of a bacterial host in media comprising annexin, e.g. from about 0.01 mg/ml to about 1 mg/ml; or around 0.1 mg/ml annexin, and from about 1 mM to about 5 mM Ca⁺⁺. Infection can be performed in liquid media, on plates, etc. as known in the art. Infection on plates may provide for an initial high concentration of phage.

Media, or plaques containing phage, are suspended in a solution comprising annexin, e.g. from about 0.01 mg/ml to about 1 mg/ml; or around 0.1 mg/ml annexin, and from about 1 mM to about 5 mM Ca⁺⁺. The solution is filtered to separate phage from bacteria, e.g. through a 0.22 μm syringe filter, and resuspended in annexin/calcium solution. The suspension may be further processed by centrifugation, etc. to reduce bacterial particulates.

The resulting phage suspension is digested with DNAse for a period of time sufficient to reduce free DNA in the suspension, e.g. from about 10 minutes to about 1 hour. The resulting suspension can be washed and concentrated, for example and without limitation in a series of spin cycles with resuspension in the annexin solution after each cycle. The number of spin cycles may be at least 1, at least 2, at least 3, at least 4, at least 5, at least 6, at least 7, and up to about 15, up to about 12, up to about 10. After a final centrifugal concentration, the filtered phage/annexin/Ca⁺⁺ solution can be stored for future use.

The concentration of phage in the suspension can be determined by any convenient method, as known in the art. For example, plaque titration assays can be used to determine phage titer. For a review of methods, see, for example, Anderson et al., (2011) Bacteriophage 1(2):86-93, herein specifically incorporated by reference.

For therapeutic applications, the high concentration phage formulation is administered to a mammal, preferably a human, in a physiologically acceptable dosage form, including those that may be administered to a human intravenously as a bolus or by continuous infusion over a period of time. Alternative routes of administration include topical, intramuscular, intraperitoneal, intra-cerobrospinal, subcutaneous, intra-articular, intrasynovial, intrathecal, oral, topical, or inhalation routes. The high concentration phage formulation also is suitably administered by intratumoral, peritumoral, intralesional, or perilesional routes or to the lymph, to exert local as well as systemic therapeutic effects.

Pharmaceutical formulations can include, depending on the formulation desired, pharmaceutically-acceptable, non-toxic carriers of diluents, which are defined as vehicles commonly used to formulate pharmaceutical formulations for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical formulation can include other carriers, adjuvants, or non-toxic, nontherapeutic, nonimmunogenic stabilizers, excipients and the like. The formulations can also include additional substances to approximate physiological conditions, such as pH adjusting and buffering agents, toxicity adjusting agents, wetting agents and detergents.

The formulation can also include any of a variety of stabilizing agents, such as an antioxidant for example. When the pharmaceutical formulation includes a polypeptide, the polypeptide can be complexed with various well-known compounds that enhance the in vivo stability of the high concentration phage formulation, or otherwise enhance its pharmacological properties (e.g., increase the half-life of the formulation, reduce its toxicity, enhance solubility or uptake). Examples of such modifications or complexing agents include sulfate, gluconate, citrate and phosphate. The high concentration phage formulation can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical formulations can be administered for prophylactic and/or therapeutic treatments. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀ Compounds that exhibit large therapeutic indices are preferred.

The data obtained from cell culture and/or animal studies can be used in formulating a range of dosages for humans. The dosage of the active ingredient typically lines within a range of circulating concentrations that include the ED₅₀ with low toxicity. The dosage can vary within this range depending upon the dosage form employed and the route of administration utilized.

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

The active ingredient, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical formulations are preferably of high purity and are substantially free of potentially harmful contaminants (e.g., at least National Food (NF) grade, generally at least analytical grade, and more typically at least pharmaceutical grade). Moreover, formulations intended for in vivo use are usually sterile. To the extent that a given compound must be synthesized prior to use, the resulting product is typically substantially free of any potentially toxic agents, particularly any endotoxins, which may be present during the synthesis or purification process. Formulations for parental administration are also sterile, substantially isotonic and made under GMP conditions.

The effective amount of a therapeutic formulation to be given to a particular patient will depend on a variety of factors, several of which will be different from patient to patient. A formulation may be provided, for example, in a unit dose. A competent clinician will be able to determine an effective amount of a therapeutic agent to administer to a patient. Dosage of the surrogate will depend on the treatment, route of administration, the nature of the therapeutics, sensitivity of the disease to the therapeutics, etc. Utilizing LD₅₀ animal data, and other information available, a clinician can determine the maximum safe dose for an individual, depending on the route of administration. Formulations which are rapidly cleared from the body may be administered at higher doses, or in repeated doses, in order to maintain a therapeutic concentration. Utilizing ordinary skill, the competent clinician will be able to optimize the dosage of a particular therapeutic or imaging formulation in the course of routine clinical trials. Typically the dosage will be 0.001 to 100 milligrams of agent per kilogram subject body weight.

The formulations can be administered to the subject in a series of more than one administration. For therapeutic formulations, regular periodic administration (e.g., every 2-3 days) will sometimes be required, or may be desirable to reduce toxicity. For therapeutic formulations which will be utilized in repeated-dose regimens, moieties which do not provoke immune responses are preferred.

In another embodiment of the invention, an article of manufacture containing materials useful for the treatment of the conditions described herein is provided. The article of manufacture comprises a container and a label. Suitable containers include, for example, bottles, vials, syringes, and test tubes. The containers may be formed from a variety of materials such as glass or plastic. The container holds a formulation that is effective for treating the condition and may have a sterile access port (for example the container may be an intravenous solution bag or a vial having a stopper pierceable by a hypodermic injection needle). The active agent in the formulation is the high concentration phage formulation (i.e. high concentration phages stocks/cultures comprising annexin). The label on, or associated with, the container indicates that the formulation is used for treating the condition of choice. Further container(s) may be provided with the article of manufacture which may hold, for example, a pharmaceutically-acceptable buffer, such as phosphate-buffered saline, Ringer's solution or dextrose solution. The article of manufacture may further include other materials desirable from a commercial and user standpoint, including other buffers, diluents, filters, needles, syringes, and package inserts with instructions for use.

As used herein, the term “therapeutically effective amount” means an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, stabilizes one or more characteristics of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment.

For example, in some embodiments, term “therapeutically effective amount”, refers to an amount which, when administered to an individual in need thereof in the context of inventive therapy, will block, stabilize, attenuate, or reverse a disease process occurring in said individual.

Formulations to be used for in vivo administration are typically sterile. Sterilization of the formulations of the present invention may readily accomplished by filtration through sterile filtration membranes.

Typically, formulations are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249:1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient. The pharmaceutical formulations are generally formulated as sterile, substantially isotonic and in full compliance with all Good Manufacturing Practice (GMP) regulations of the U.S. Food and Drug Administration.

Also provided are kits for use in the methods. The subject kits include a high concentration phage formulation. In some embodiments, the components are provided in a dosage form (e.g., a therapeutically effective dosage form), in liquid or solid form in any convenient packaging (e.g., stick pack, dose pack, etc.). Reagents for the selection or in vitro derivation of cells may also be provided, e.g. growth factors, differentiation agents, tissue culture reagents; and the like.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a removed site.

### EXPERIMENTAL

Annexin V is a safe enzymatically inert human protein that binds to Lipid A/lipopolysaccharide (LPS) bacterial membrane contaminants and negatively charged chemical species (i.e. N-glycans) within the phage protein coat, to prevent aggregation, simplify purification and stabilize phage preparations at high enough phage titer (>10¹²/ml) to directly lyse target bacteria following administration in contrast to low titer (10⁷ to 10⁸/ml) phage infection/replication protocols used in clinical trials which often require weeks of daily injections. Furthermore, bacterial growth is generally inhibited in vivo limiting the speed and efficacy of low titer phage therapies even against susceptible bacteria. An alternative paradigm is based on the concept of lysis from without. High local concentrations of phage are capable of direct bacterial lysis completely bypassing or dramatically reducing the need for bacterial replication in vivo. Stabilization of phage suspensions with annexin V also permits long-term storage, easy shipment, and the creation of libraries/repositories of clinically relevant classes/strains of bacteria immediately ready for injection. Stabilization of phage preparations with annexin V at high titer also opens the door to radiolabeling and radionuclide imaging/biodistribution studies of phages and sites of bacterial infection in patients with antibiotic resistant infections.

Although lysis from without is expected to be rare and uncommon in vivo with standard phage injection densities of 10⁷-10⁸ pfu/ml, we have found that a single dose of phage at concentrations >10¹² pfu/ml can rapidly reverse lethal bacterial wound infections in mice. The advantages of direct phage lysis include, speed, reduced chance of resistant gene transmission, avoidance of known bacterial phage resistance mechanisms, elimination of interphage interference, and the potential increase in phage cross-reactivity, which could broaden bacterial coverage without the use of phage cocktails. However, there are no generalizable methods to consistently generate highly concentrated (>10¹² pfu/ml), contaminant-free bacterial phage suspensions that are stable in solution without self-aggregation or loss of viability for systemic injection.

Previously, we purified and concentrated bacterial lysates in preparation for radiolabeling through a combination of DNase I treatment followed by multiple filtration steps and demonstrated that this process reduced phage precipitation and self-aggregation in solution. Although this protocol was successful for PAML31-1 short-tailed P. aeruginosa (PA) phage, we could not consistently achieve phage titers greater than 10⁷-10⁸ pfu/ml without adding low amounts of annexin V (0.1 mg/ml) for E. coli short-tailed A phage strains. Annexin V is an enzymatically inert endogenous human protein that has been extensively studied in multiple clinical imaging trials. This protein selectively and reversibly binds to phosphatidylserine and other negativity charged molecules with nanomolar affinity in the presence of ionized calcium. Interestingly, annexin V can also bind to Lipid A and phosphatidylethanolamine, both of which are integral membrane components of gram-negative bacteria. Indeed, systemic injection of annexin V can prevent or reverse the toxic effects of LPS in multiple animal models and is now in early clinical trials for the treatment of sepsis. Studies have also shown that annexin V specifically binds certain negatively charged N-glycan chemical species that are known constituents of phage capsids and tails. The stability of filamentous phages or those with a relatively large number of negative charges exposed on their tails is enhanced with small amounts of annexin V due to its ability to shield negatively charged chemical species known be exposed on the surface of most if not all phages. This stabilization overcomes issues that limit the widespread clinical use of phage and allow clinical trials investigating the use of phage treatment for PA, E. coli, and other common bacterial infections.

Not only does the use of annexin V in phage solution lead to stabilized of phage stocks it also allows for reduced bacterial contamination. This is achieved through sequential washing with annexin V because annexin V removes bacterial contaminants from both the solution and the phage itself through binding to bacterial LPS and other bacterial membrane lipids. Treatment with DNAse I further reduces contamination of stabilized phage solutions by degrading free bacterial DNA leading to high purity stabilized phage solutions.

We first investigated the biodistribution of phage injected intravenously (IV). An early study reported that ⁵¹Cr-labeled T4 λ phage immediately localized to the liver, while another study found that radiolabeled λ phage primary localized to the spleen followed by delayed clearance. We found that the presence of bacterial DNA in our Xen5 lysates not only resulted in high liver and spleen uptake of ⁹⁹mTc-HYNIC-labeled PAML31-1 phage with delayed blood clearance but also could not be removed even with multiple washing cycles. However, treating bacterial lysates with DNAse I prior to phage purification, HYNIC conjugation and radiolabeling decreased the high non-specific uptake by the reticuloendothelial system in the liver and spleen and improved blood clearance of radiolabeled phage. DNAse I treatment likely prevented the formation of phage/bacterial DNA colloids, presumably the source of the high liver and spleen uptake and slow blood clearance seen in prior investigations. However, the 2 hour reaction time recommended for protein conjugation reduced phage viability by more than 90%. Fortunately, a brief 3-min reaction with HYNIC immediately followed by quenching preserved phage viability.

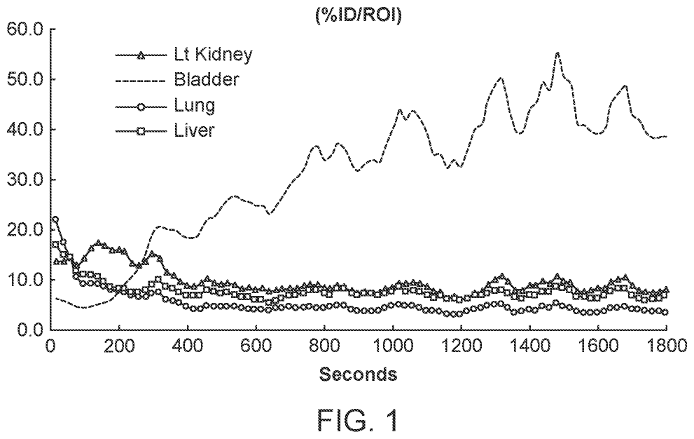

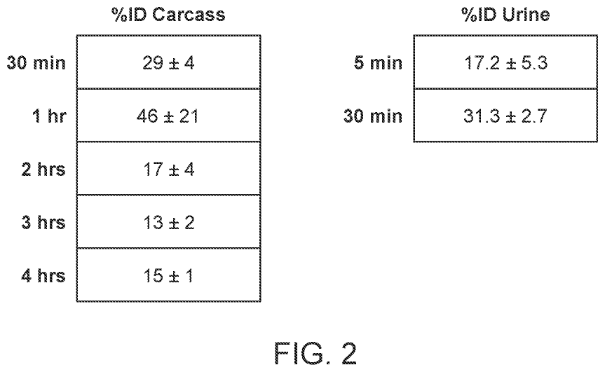

After optimizing phage purification and HYNIC conjugation, we performed dynamic radionuclide imaging and biodistribution assays after IV bolus injection of radiolabeled phage. Dynamic imaging showed a very rapid urinary excretion of tracer with visible bladder activity occurring within 200 seconds after bolus injection (FIG. 1). Total urinary excretion at 5 and 30 min was 17.2±5.3% and 31.3±2.7% of injected dose (% ID), respectively (FIG. 2). Urinary phage concentrations of >10⁸ pfu/ml confirmed the presence of viable phage, though at far smaller quantities than expected based on measurements of excreted ⁹⁹mTc activity. This was likely due to the adverse effects of low pH in the urine on phage viability. While previous studies noted significant urinary excretion of tracer 30, the speed of onset (i.e., starting within 200 s after injection) was a new finding. The rapid appearance of activity within the bladder was unlikely to be a result of renal metabolism of labeled phage or the ^{99m}Tc-HYNIC moiety, which when cleaved from proteins within renal tubular epithelial cells is effectively trapped within the kidney. These data strongly suggest a conserved mechanism(s) for the rapid transit of phage across vascular endothelium far faster than any previously described in the literature.

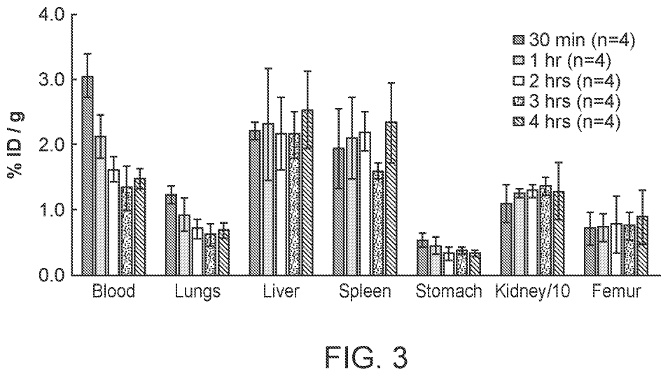

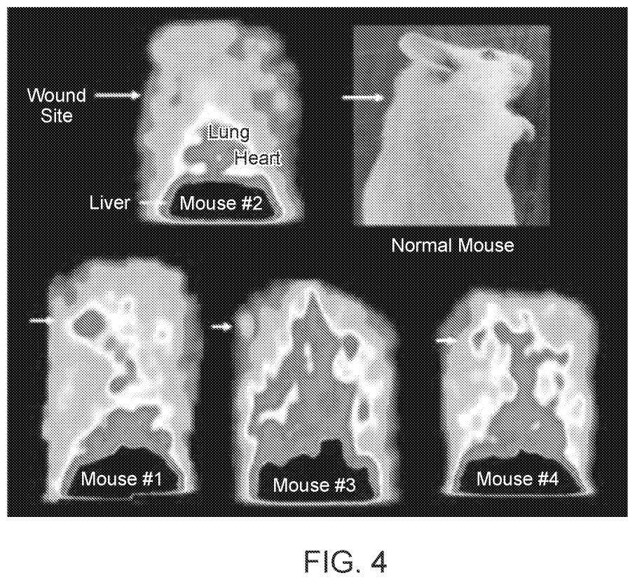

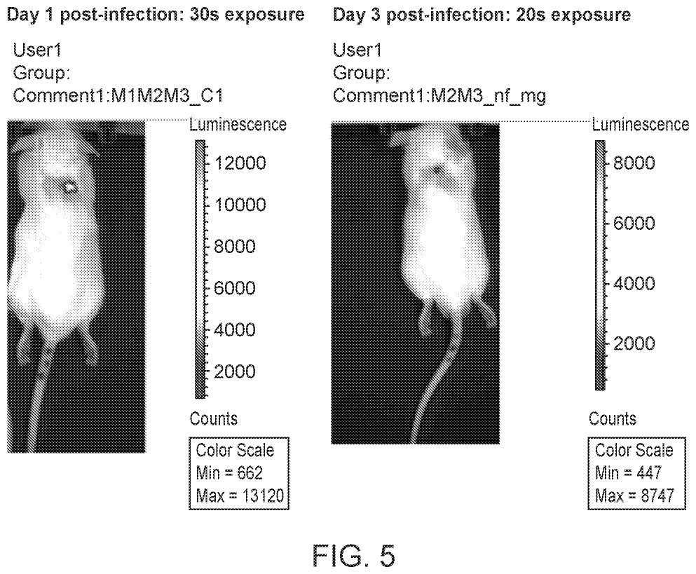

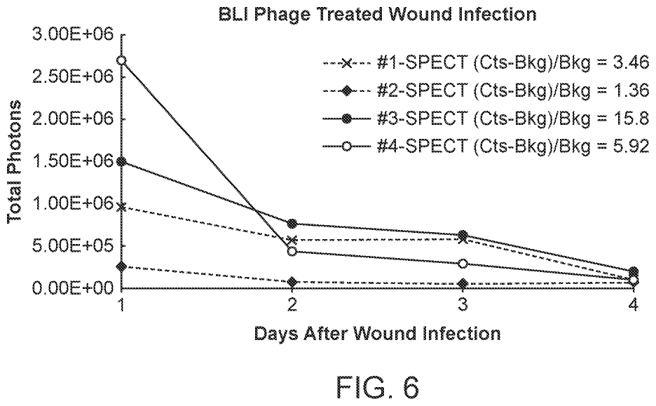

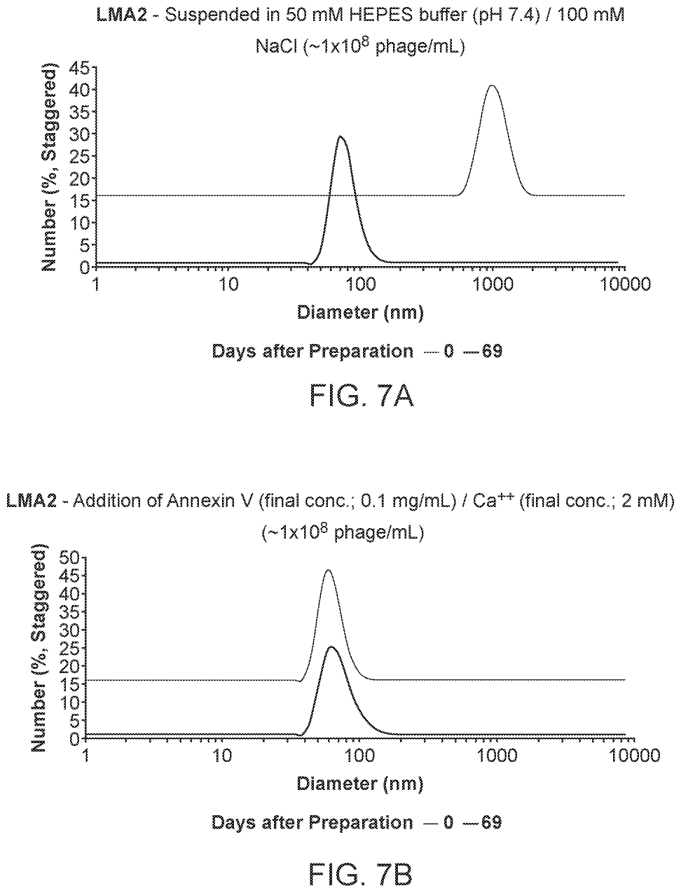

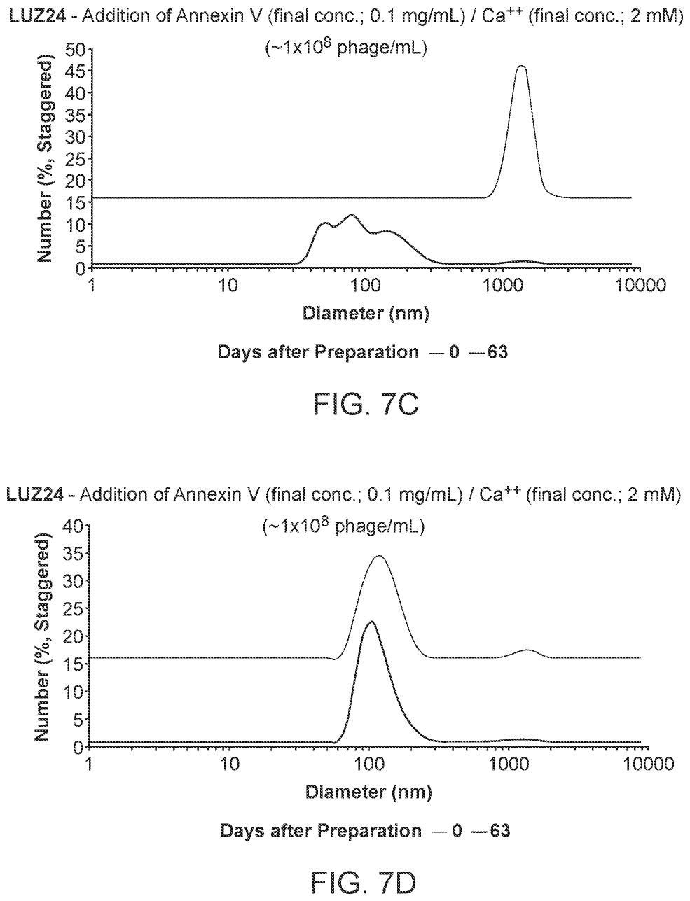

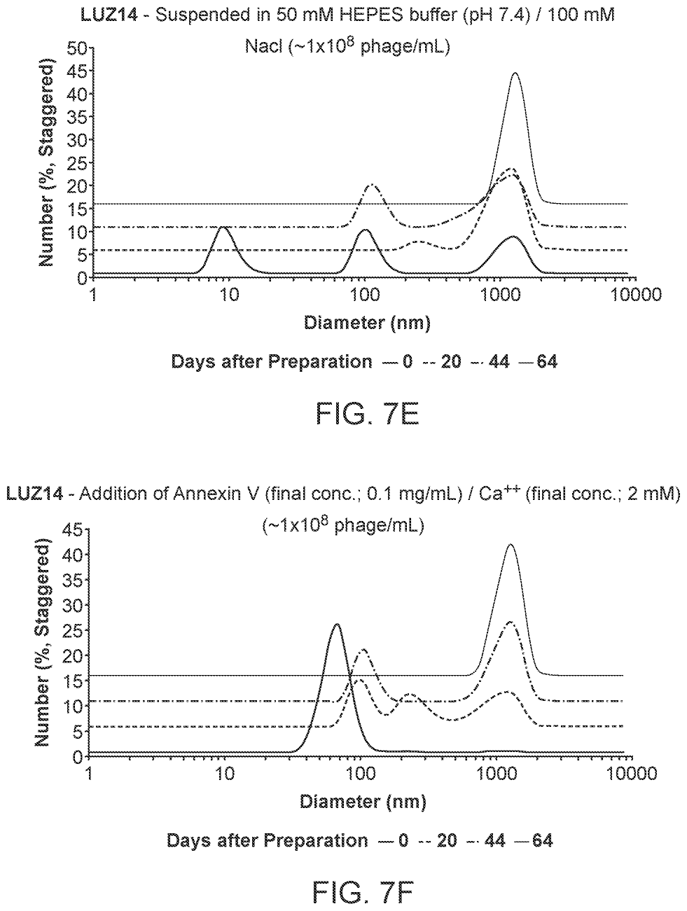

Biodistribution assays at 30 min and 1, 2, 3, and 4 h as shown in FIG. 2 revealed a marked wash-in and washout of activity in the carcass, peaking at 46% of injected activity at 1 hour and dropping to 17% at 2 hour. FIG. 3 demonstrates a rapid clearance of ⁹⁹mTc-HYNIC phage from the blood and lungs, while the uptake of radiolabeled phage in other major organs, including the liver, spleen, kidneys, and bone marrow (femur) remained relatively constant after 30 min. The marked redistribution of tracer activity in the carcass in normal mice over a period of 2 h demonstrates that the majority of phage injected IV can rapidly spread throughout the soft tissues of the body in the extracellular space without being trapped intracellularly. As with the kidney, phages easily cross vascular endothelium throughout the body (with the exception of the brain) in a fashion similar to proteins that are filtered by the kidneys (<60 kDa) despite their large size (≈1-3 mDa). Imaging and Treatment of Lethal Pseudomonas Wound Infection with Radiolabeled Phage. To demonstrate the in vivo infectivity of ^{99m}Tc-HYNIC-phage, we injected four young adult male CD-1 mice with 20 MBq (250 μL volume, 9.38×10¹⁴ labeled phage per dose) of radiolabeled phage 1 day after wounding and inoculating with 1×10⁶ Xen5 PA bacteria. Two hours after injection of tracer, sedated mice underwent SPECT imaging as shown in FIG. 4. All mice survived another 4 days without clinical signs of infection. This result is particularly remarkable as the bacterial dose used in this animal model results in the death of most infected mice within 5 days.

### Methods

Phage propagation and purification with Annexin V/CaCl₂ solution. Annexin V (0.1 mg/ml)/CaCl₂ (2 mM) solution was added to LB (Luria-Bertani) agar the night before steaking with bacteria to grow a bacterial lawn for lambda phage propagation. In some embodiments, the bacterial strain is CRM1 E. coli. In other embodiments, the bacterial strain includes but is not limited to, PAO1 P. aeruginosa, Xen5 P. aeruginosa, FRI1169 S. aureus, etc. 10 uL of phage was added to Annexin V (0.1 mg/ml)/CaCl₂ (2 mM) solution followed by overnight propagation at 37° C. in order to generate lytic plaques on the bacterial lawn. In some embodiments, the phage used is W60 Icl60cY17 (NIH stock) lambda phage. In other embodiments, the phage used includes but is not limited to, PF1, Pf4, Fd1, W60, PAML-31-1, etc. Lytic plaques were excised from agar plate and they placed into Annexin V (0.1 mg/ml)/CaCl₂ (2 mM) solution. The plaques were then finely minced by razor blade and passed through a 0.22 μm syringe filter filled with 15 ml of fresh Annexin V (0.1 mg/ml)/CaCl₂ (2 mM) solution. 15 ml of filtered phage/annexin V/Ca⁺⁺ solution was then treated for 30 minutes with 20 mg of DNAse at 37° C. Next, a series of 15 spin cycles with a Millipore-Amicon Ultra-15 100 kDa 15 ml centrifugal filter spun down to a volume less than 1 ml and diluted back to 15 ml with fresh Annexin V (0.1 mg/ml)/CaCl₂ (2 mM) solution each cycle). After 100 kDa of centrifugal purification, the filtered phage/annexin V/Ca⁺⁺ solution (10 ml) was stored at 4° C.

Plaque titration assay. Serial dilutions of phage using fresh Annexin V (0.1 mg/ml)/CaCl₂ (2 mM) solution were made and 10 μL of each were plated on a CRM1 bacterial lawns at 37° C. and were incubated over night to determine phage titer. Plate titer was found to be 10¹¹ per 10 μL of phage preparation or 10¹³ pfu/ml. Without the addition of annexin V/Ca⁺⁺ we were consistently unable to concentrate phage beyond 10⁸ pfu/ml without its aggregation and precipitation in solution after overnight storage at 4° C.

Measurement of phage aggregation. Samples of LMA2/Myoviridae, PB-1-like (A & B), LUZ24/Podoviridae, T7-like (C & D) and LUZ14/Podoviridae, T7-like (E & F) short tailed phages capable of infecting and lysing clinically isolated common strains of Pseudomonas aeruginosa (Ceyssens et al 2009; Ref A below) were grown, underwent DNAse I treatment and filtration into 50 mM HEPES (pH-7.4)/100 mM NaCl with or without Annexin V (0.1 mg/ml, final concentration)/Ca2+(2 mM, final concentration) and at a density of 10¹² to 10¹³ phage per ml as previously described (Holman et all 2017, Ref B below). These phage strains were used as part of a phage cocktail to treat burn patients in a recent clinical trial (Jault et al 2019). The cocktail made up of 12 natural lytic anti-P. aeruginosa bacteriophages, known as PP1131 while demonstrating clinical efficacy as topical agent had a significantly longer time to achieve bacterial control than standard topical antibiotic treatments, directly related to the loss of phage during GMP manufacturing resulting a huge reduction of phage concentration, which resulted in 1,000-fold to 10,000-fold lower dose of active phage. The instability of phage during preparation and short-term storage was defined as one of key obstacles to overcome for the further development of clinically effective phage therapy (Huang et al. 2019).

Dynamic light scattering (DLS) was used to determine the size distribution profile of phage virions in HEPES/NaCl buffer with or without the addition of annexin V/Ca⁺⁺ as described above. Phage aggregation were demonstrated with an average diameter of ˜1000 nM whereas single phage particles had an average diameter of ˜100 nM. Note before interrogation phage samples were diluted in matching buffer to a concentration of ˜1×10⁸ phage/ml to avoid saturation of the DLS detector. 11 separate acquisitions per sample were obtained to average out changes in scattering due to random motion of phage particles/orientation in buffer. Samples were stored long term at 4° C. under sterile conditions. A) demonstrates complete aggregation of LMA2 phage after 69 days of storage buffer without annexin V/Ca⁺⁺ whereas B) showed no evidence of aggregation at Day 69. C) shows early aggregation with a broad range of LUZ24 phage/aggregate sizes even on Day 0 followed by complete aggregation at Day 69. D) demonstrates that annexin V/Ca⁺⁺ prevented the LUZ24 phage aggregation at Day 0 and at Day 69. E) shows early aggregation with a broad range of LUZ14 phage/aggregate sizes at Day 0 followed by complete aggregation at Day 69, while in F) annexin V/Ca⁺⁺ prevented LUZ14 aggregation at Day 0 and reduced the amounts of aggregation at Day 20 as compared to E).

Test if the addition of annexin V improves the purity and stability of phage preparations. We propagate phage to titers of 10⁷-10⁸ pfu/ml. In some embodiments, the phage used include but are not limited to, PF1, Pf4, Fd1, W60, PAML-31-1, etc. We treat bacterial lysates in 15 ml flasks with 20 mg of DNase I at 37° C. for 3 hours followed by 3 washes with ×1 phosphate buffered solution (PBS) using Amicon Ultra-100 kDa 15 ml centrifugal filters. For annexin V preparations we wash and resuspend in a solution of 0.1 mg/ml of annexin V in 2 mM CaCl₂ mixed with ×1 PBS. Titered phage solutions are then tested for component purity with FTIR spectromicroscopy by taking advantage of the coffee-ring effect. We quantify the amount of endotoxin in each preparation in triplicate with the ETOXATE™ (Sigma) kit. Plaque assays determine phage viability and stability at 1, 3, 7, 14 and 30 days after preparation. We then repeat these experiments with the goal of reaching phage densities greater than 10¹² pfu/ml. It may not be possible to reach high densities without annexin V during preparation and storage. We made three attempts to concentrate and test PF1, Pf4, Fd1 and W60 without annexin V before concluding failure.

Define annexin V binding affinity for phage. We perform a Scatchard dilutional assay using 500 μL aliquots of phage (10⁷-10⁸ pfu/ml) in a saturating solution of annexin V [0.1 mg/ml (278 nM) protein in 2 mM CaCl₂ mixed with ×1 PBS]. Triplicate samples are incubated with 0, 0.01, 0.05, 0.1, 0.5, 1.0, 5.0, 10, 50, and 100 nM of ^{99m}Tc-HYNIC annexin V, prepared according to Blankenberg et al., for 1 hour at room temperature. We doubly wash samples with 2 mM CaCl₂/×1 PBS buffer with 100 kDa centrifugal filters. We measure phage bound activity using a gamma counter (corrected for decay) and then calculate the binding constant (kd) of annexin V for each phage strain.

Define the pharmacokinetics of Tc^{99m} labeled phage via dynamic imaging and biodistribution assay. Preparation of Radiolabeled Phage—We conjugate phage/annexin V preparations with densities >10² pfu/ml, the minimum needed to for radionuclide imaging, with HYNIC. Next, we label the preparations with ^{99m}Tc as described by Holman with the following modifications, a) HYNIC-annexin V removed from HYNIC-phage after quenching, by three cycles of 100 kDa centrifugal filtration with 115 mM Tricine (pH6.8) buffer containing 0.1 mg/ml of unlabeled annexin V, 2 mM CaCl₂, and 100 mM NaCl and b) purified phage conjugates remain in the buffer solution until chelation of aqueous ^{99m}Tc using the tin-tricine reaction agent. After ^{99m}Tc chelation, we purify radiolabeled phage and elute it into a solution of 0.1 mg/ml of unlabeled annexin V, 2 mM CaCl₂, and 140 mM NaCl using a NAP-5 desalting column (GE Healthcare™)

Radionuclide Imaging and Biodistribution Assays—We inject young adult CD-1 mice with 100 to 300 μCi of ^{99m}Tc-HYNIC-phage, IV. Groups of 5 mice are euthanized for biodistribution assay at 0.5, 1, 2 and 4 hours after injection of radiolabeled phage. Mice from the 0.5 hours group are injected and serially imaged (2 s frames×5 min, then 20 s frames×15 min) over 20 min followed by a 10 min static image (parallel hole collimator, 128×128 matrix) using the A-SPECT small animal imaging unit in the Blankenberg Lab. Static images are also be obtained over 10 min for the 1, 2 and 4 hours groups just prior to euthanization. The lungs, liver, spleen, kidneys, as well as blood, urine and thigh muscle samples are removed, weighed and assayed for ^{99m}Tc activity with a gamma counter. We express results as % ID and % ID/gram corrected for decay. After removing all organs and fluids, we place carcasses into a clean disposable glove and assay retained ^{99m}Tc activity using a dose calibrator and express results as % ID.

Fluorescent Labeling of Phage—We use Cy5.5-NHS ester (Lumiprobe™) kits to labeled purified phage (i.e., in solutions containing 0.1 mg/ml of annexin V and 2 mM CaCl₂ at a density greater than 10¹² pfu/ml) following the manufacturer's protocol with the following modifications; a) as with the HYNIC-phage conjugation reaction time is limited to 3 min before quenching in order to preserve phage viability, and b) Cy5.5-annexin V is removed from Cy5.5-labeled phage after conjugation and quenching, by three cycles of 100 kDa centrifugal filtration with a solution of 0.1 mg/ml of unlabeled annexin V, 2 mM CaCl₂, and 140 mM NaCl.

Confocal laser scanning microscopy (CLSM) of peripheral blood and histologic sections of kidney, lung, liver, spleen, and muscle—We co-inject Cy5.5-NHS-phage with equimolar amounts of ^{99m}Tc-HYNIC-phage. After euthanization samples of lung, liver, spleen, kidney, and thigh muscle are placed in Optimal cutting temperature compound (OCT) and flash frozen on dry ice and stored at −80° C. prior to histologic sectioning. We obtain samples of citrated whole blood and store at 4° C. prior to staining and confocal microscopy. We cut 10⁻²⁰ μm transverse sections of kidney and non-anatomically orientated sections of lung, liver, spleen and thigh muscle with a cryomicrostat located in the Blankenberg Lab. The PKH67 green fluorescent lipid membrane marker (PhanosTechnologies, Sigma-Aldrich) serve as a counter stain to outline plasma membranes in histologic sections. Lung, liver and spleen samples is also counter stained with fluorescent blue anti-CD68 antibody to mark resident tissue/alveolar macrophages. After counter staining, we image sections at the Beckman Center with a Leica SP8 White Light Confocal, gSTED, FLIM microscope (Cell Sciences Imaging Facility, Stanford). We analyze intact (non-cut) cells within central optical tomographic stacks. We count the number of Cy5.5 labeled phages within and outside cells in 20 random high-powered 40× fields. We also calculate the average number of phages within tissue macrophages. Whole blood smears are stained with PKH67 and appropriate fluorescent antibodies to mark T and B cells, monocytes, and neutrophils. We calculate the average number of phages within each phenotype and in plasma per high powered field.

Determine phage delivery and efficacy of intraperitoneal (IP) infusion vs. bolus injection in Xen5 infected wounds. We perform baseline BLI on Day 1 after inoculating wounds with Xen5. We then begin phage therapy in separate groups of 5 mice via IP injection or infusion of ˜10¹³ EdU-5 phage/day. In some embodiments, the phage used include but are not limited to, PF1, Pf4, Fd1, W60, PAML-31-1, etc. Mice undergo daily BLI to monitor bacterial burden prior to euthanization on Days 2, 3, 4 or 5 for biodistribution assay of EdU-5-labeled phage including, infected wound, blood, urine, lung, liver, spleen, kidney and skeletal muscle after tissue homogenization. Samples are weighed and EdU-phage number determined by EdU fluorescence using the Click-iT® kit (ThermoFischer) according to manufacturer's instructions. We express results as the number of punctate fluorescent foci per organ (tissue) and per gram of tissue.

PA Wound Model. The upper backs of young adult male CD-1 mice (8-10 weeks) are shaved under anesthesia with 2-2.5% isoflurane as described previously. Next a single 3-5 mm cutaneous slit wound is made in the midline, and a pocket created just under the skin using a pair of sterile scissors. 20 μL of PBS containing 10⁶ Xen5 bioluminescent bacteria are then added to the wound with a pipet tip. Wounds are sealed with Vetbond Tissue Adhesive (3 M #70200742529).

BLI of Infected Mice. Infected mice are anesthetized with 1-2% isoflurane using the SAS3 anesthesia system. We then image mice in an IVIS Spectrum unit, with 30 s exposures using the “small” binning. We analyze images for bacterial wound luminescence with Living Image software (Caliper Life Sciences).

Preparation of EdU-5 labeled Phages. We propagate phages in the presence of 1-10 μM of 5-EdU (5-ethynyl-2′-deoxyuridine) during logarithmic bacterial growth as described by Ohno et al. After purification, EdU-5 labeled phage preparations are titered by plate assay. We determine the average amount of EdU-5 incorporated into phage using the EdU-5 fluorescence Click-iT® kit with results expressed as mean fluorescence/pfu for punctate fluorescent foci. This value is used to calculate the number of phages present in homogenized tissue samples obtained from biodistribution assays as described above.

Osmotic Pump and IP Catheter Placement. Mice are anesthetized with 2-2.5% isoflurane. The mid abdomen is shaved and prepped with three Povidone Swabsticks followed by a rinse with 70% isopropyl alcohol. After drying, the upper abdominal region is sterilely draped. A 2-3 mm vertical midline incision is made in the sub-xiphoid region through which an Alzet intraperitoneal catheter (#0007771) is inserted into the peritoneal cavity. We close the muscular incision using a 4-0 Vicryl purse string suture around the exiting catheter. The exposed catheter is subcutaneously tunneled to the lower back and attached to a 1003D pump filled with sterile saline. Skin incisions is closed with 9 mm staples. Animals are recovered for two days prior to wounding on Day 0. After Day 1 BLI we replace the saline pumps with either 1001D or 1003D pumps filled with phage using sterile technique.

Determine efficacy of W60 and Argo1 after IP injection into mice with CRM1 urinary tract infections. We inject young adult male CD-1 mice (8-10 weeks) with 10⁷ cfu of CRM1 uropathic E. coli in 50 μL 1×PBS into the bladder via urethral a catheterization with a Pediatric 24G yellow (¾″) IV access cannula. We prepare EdU labeled W60 and Argo1 phage as described in above and inject (≈10¹³ pfu/day ingroups of 10 mice. We euthanize separate groups of mice for phage biodistribution assay (as described above) and urinary tract cultures on Days 2, 4, and 7 after CRM1 inoculation. We also fix bladder and kidney samples in formalin. We embed fixed tissue samples in paraffin and prepare 6 to 7 μm histologic sections, deparaffinize, and stain with hematoxylin and eosin. We then grade the severity of urinary tract infection using the methods of Li et al.

### REFERENCES

A) Ceyssens P J, Noben J P, Ackermann H W, Verhaegen J, De Vos D, Pirnay J P, Merabishvili M, Vaneechoutte M, Chibeu A, Volckaert G, Lavigne R. Survey of Pseudomonas aeruginosa and its phages: de novo peptide sequencing as a novel tool to assess the diversity of worldwide collected viruses. Environ Microbiol. 2009 May; 11(5):1303-13. doi: 10.1111/j.1462-2920.2008.01862.x. Epub 2009 February 4. PMID: 19207572
   B) Derek Holman 1, Matthew P Lungren 2, Jonathan Hardy 3, Chris Contag 1 2 3, Francis Blankenberg. Preparation of Tc99m-Labeled Pseudomonas Bacteriophage without Adversely Impacting Infectivity or Biodistribution. Bioconjug Chem. 2017 Nov. 15; 28(11):2698-2706. doi: 10.1021/acs.bioconjchem.7b00395. Epub 2017 Oct. 26 PMID: 29020448
   C) Jault P, Leclerc T, Jennes S, Pirnay J P, Que Y A, Resch G, Rousseau A F, Ravat F, Carsin H, Le Floch R, Schaal J V, Soler C, Fevre C, Arnaud I, Bretaudeau L, Gabard J. Efficacy and tolerability of a cocktail of bacteriophages to treat burn wounds infected by Pseudomonas aeruginosa (PhagoBurn): a randomised, controlled, double-blind phase 1/2 trial. Lancet Infect Dis. 2019 01; 19(1):35-45. PMID: 30292481
   D) Huang G, Wei Z, Wang D. What do we learn from the “PhagoBurn” project. Burns. 2019 February; 45(1):260. doi: 10.1016/j.burns.2018.11.008. Epub 2018 Dec. 5. PMID: 30527641

1. A pharmaceutical formulation comprising:
an effective dose of annexin protein and a high concentration of phage,
wherein the presence of the annexin protein reduces bacterial contamination and phage aggregation relative to a pharmaceutical formulation lacking annexin to produce a stabilized phage formulation.

2. The pharmaceutical formulation of claim 1, wherein the phage is a filamentous phage.

3. The pharmaceutical formulation of claim 1, wherein the annexin used is human annexin V.

4. The pharmaceutical formulation of claim 1, wherein an effective dose of annexin is 0.01 mg/ml to 1 mg/ml.

5. The pharmaceutical formulation of claim 1, wherein the annexin protein is a wild-type protein.

6. The pharmaceutical formulation of claim 1, wherein the annexin protein comprises at least one amino acid substitution relative to the wild-type protein.

7. The pharmaceutical formulation of claim 1, wherein the annexin protein has at least 90% sequence identity to SEQ ID NO: 1.

8. The pharmaceutical formulation of claim 1, wherein bacterial contamination is reduced to at least 0.01% of the total stock.

9. The pharmaceutical formulation of claim 1, wherein phage precipitation is reduced by at least 10%.

10. The pharmaceutical formulation of claim 1, wherein the phage concentration is greater 10⁸ pfu/ml.

11. The pharmaceutical formulation of claim 1 wherein the stabilization phage formulation also contains from about 1 to 5 mM CaCl₂.

12. A method of treating a bacterial infection in a subject, the method comprising:
administering to the subject the pharmaceutical formulation of claim 1 to cause bacterial lysis to resolve the infection.

13. The method of claim 12, wherein the amount of phage administered is greater than 10⁸ pfu/ml.

14. The method of claim 12, wherein the bacterial infection is cause by a bacterium that is resistant to antibiotics.

15. The method of claim 1, wherein the bacterial infection is caused by a gram-negative bacterium.

16. The method of claim 15, wherein the gram-negative bacterium is selected from Acinetobacter baumannii, Klebsiella pneumoniae, Escherichia coli, Campylobacter jejuni, Neisseria gonorrhoeae, Salmonella Typhi, nontyphoidal Salmonella, Shigella dysenteriae, Shigella flexneri, Shigella boydii, Shigella sonnei, and Pseudomonas aeruginosa.

17. The method of claim 1, wherein the pharmaceutical formulation is administered locally at the site of infection.

18. The method of claim 1, wherein the pharmaceutical formulation is administered systemically.


SEQUENCE LISTING (1 sequence)

SEQ ID NO: 1 - protein, 320 aa, Homo sapiens
MAQVLRGTVTDFPGFDERADAETLRKAMKGLGTDEESILTLLTSRSNAQRQEISAAFKTLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGEDD